(19) United States
(12) Patent Application Publication
(10) Pub. No.: US 2026/0315382 A1
(43) Pub. Date: Oct. 15, 2026

(54) ATOMOXETINE HYDROCHLORIDE ORAL SUSPENSION AND USE THEREOF

(71) Applicant: OWP Pharmaceuticals, Inc., Naperville, IL (US)

(72) Inventors: Paul SUDHAKAR, Shawnee, KS (US); Scott BOYER, West Chicago, IL (US)

(21) Appl. No.: 19/179,733

(22) Filed: Apr. 15, 2025

(51) Int. Cl. A61K 9/10; A61K 9/00; A61K 31/138; A61K 47/02; A61K 47/10; A61K 47/12; A61K 47/14; A61K 47/20; A61K 47/22; A61K 47/26; A61K 47/38; A61K 47/46

(52) U.S. Cl. CPC A61K 9/10; A61K 9/0053; A61K 9/0095; A61K 31/138; A61K 47/02; A61K 47/10; A61K 47/12; A61K 47/14; A61K 47/20; A61K 47/22; A61K 47/26; A61K 47/38; A61K 47/46

(57) ABSTRACT

An oral liquid suspension comprising atomoxetine or a pharmaceutically acceptable salt thereof is disclosed, formulated at a concentration of about 20 mg/mL for the treatment of attention-deficit/hyperactivity disorder (ADHD). The suspension includes a dual preservative system (methylparaben and sodium benzoate), sweeteners, pH buffers, suspending agents, co-solvents, flavorants, and colorants. The composition demonstrates bioequivalence to Strattera® capsules, robust antimicrobial efficacy meeting USP <51> criteria, and physicochemical stability over 24 months in validated high-density polyethylene containers with child-resistant closures. The 20 mg/mL concentration enables precise dose titration for pediatric and dysphagic patients. Palatability is optimized through validated flavor masking studies, supporting high patient adherence. The invention further provides scalable manufacturing methods, quality control specifications, and methods of use for treating ADHD, offering a stable, safe, and patient-friendly alternative to solid oral dosage forms.

### BACKGROUND OF THE INVENTION

Atomoxetine hydrochloride, a selective norepinephrine reuptake inhibitor, has long faced formulation challenges in pediatric and dysphagic ADHD populations due to its poor palatability, dose titration limitations, and physicochemical instability in liquid forms. Prior art suspensions typically relied on single preservative systems (e.g., sodium benzoate) that precipitated crystallization at pH >6.5, compromising microbial efficacy and limiting shelf-life to ≤12 months. These formulations exhibited inadequate redispersibility (≤35% post-sedimentation) and pseudoplastic properties, requiring aggressive shaking to prevent API stratification—a critical barrier to adherence in pediatric use. Furthermore, existing solutions often fail to effectively address atomoxetine's intrinsic bitterness and tongue numbness, with taste-masking efforts using, e.g., high sorbitol/glycerin ratios, inadvertently increase water activity (a_{ω}≥0.95), fostering fungal growth. Prior art atomoxetine suspensions with high sorbitol/glycerin ratios inadvertently raised the water activity, compromising stability despite the presence of preservatives.

### SUMMARY OF THE INVENTION

The present invention resolves these limitations across specific embodiments through a dual preservative system (e.g., methylparaben:sodium benzoate, 2.5:1-4:1 w/w) that maintains USP <51>-compliant antimicrobial efficacy without crystallization at pH 7.2-7.6. By integrating PROSOLV® SMCC 50 (2% colloidal SiO₂) with xanthan gum/carboxymethylcellulose sodium, the suspension typically achieves shear-thinning viscosity (150-250 mPa·s) and ≥50% redispersibility within 30 seconds—surpassing prior art sedimentation rates. Additionally, the 22.85 mg/mL±2.5% concentration enables precise 10 mg dose increments, addressing the ≈40% dosing inaccuracies reported in weight-based pediatric regimens (0.5-1.4 mg/kg/day). These advancements overcome the stability-adherence tradeoff that plagued earlier atomoxetine liquid formulations. Humectants like sorbitol/glycerin reduce the water activity by binding water, but excessive use can paradoxically increase free water through hygroscopic effects. The invention resolves this across specific embodiments by optimizing excipient ratios (e.g., dual preservatives+PROSOLV® SMCC 50) to achieve a_{ω}≤0.92, inhibiting osmophilic fungi while maintaining taste/palatability.

The present invention provides for an oral liquid suspension that includes: atomoxetine, or a pharmaceutically acceptable salt thereof; preservative; sweetener; pH adjuster/buffer; solvent/co-solvent, suspending/thickening agent; anticaking/flow agent; flavorant; and colorant.

The present invention provides for an oral liquid suspension that includes: atomoxetine, or a pharmaceutically acceptable salt thereof; preservative (sodium benzoate, methylparaben); sweetener (saccharin sodium dihydrate, sorbitol, and sucralose); pH adjuster/buffer (sodium phosphate dibasic and sodium phosphate monobasic); solvent/co-solvent (propylene glycol, glycerin, polyethylene glycol 400, and purified water), suspending/thickening agent (carboxymethylcellulose sodium and xanthan gum); anticaking/flow agent (PROSOLV® SMCC 50 (silicified microcrystalline cellulose)); flavorant (cherry flavor); and colorant (FD&C Red No. 40 and FD&C Yellow No. 6).

The present invention provides for an oral liquid suspension that includes: atomoxetine, or a pharmaceutically acceptable salt thereof; methylparaben; sodium benzoate; saccharin sodium, dihydrate; sodium phosphate dibasic; sodium phosphate monobasic; sorbitol; propylene glycol; glycerin; PROSOLV® SMCC 50 (silicified microcrystalline cellulose); carboxymethylcellulose sodium; xanthan gum; flavoring agent, colorant; purified water; polyethylene glycol 400; and sucralose.

The present invention provides for an oral liquid suspension that includes: atomoxetine, or a pharmaceutically acceptable salt thereof; methylparaben; sodium benzoate; saccharin sodium, dihydrate; sodium phosphate dibasic; sodium phosphate monobasic; sorbitol; propylene glycol; glycerin; PROSOLV® SMCC 50 (silicified microcrystalline cellulose); carboxymethylcellulose sodium; xanthan gum; cherry flavor; FD&C Red No. 40; FD&C Yellow No. 6; purified water; polyethylene glycol 400; and sucralose.

The present invention provides for a method of treating a disease or disorder in a human, the disease or disorder ameliorated by atomoxetine, the method that includes orally administering to a human subject in need thereof the oral suspension described herein.

The present invention provides for a method of treating attention-deficit/hyperactivity disorder (ADHD) in a human, the method that includes orally administering to a human subject in need thereof the oral suspension described herein.

The present invention provides an oral liquid suspension exhibiting unexpected synergies between a dual preservative system (0.1% methylparaben+0.03% sodium benzoate) and pseudoplastic suspending agents (carboxymethylcellulose sodium/xanthan gum/PROSOLV® SMCC 50), achieving both USP <51>-compliant antimicrobial efficacy (≥3.8 log reductions against E. coli, P. aeruginosa, S. aureus, and C. albicans; ≥3.6 against B. cepacia) and 24-month physicochemical stability (pH 6.5-8.0, viscosity 150-250 mPa·s, d₉₀<300 μm) under ambient storage. Surprisingly, the formulation maintains ≥50% redispersibility after 24 hr sedimentation despite high API loading (20 mg/mL atomoxetine HCl), while demonstrating bioequivalence to Strattera® capsules with narrow 90% CIs (Cₘₐₓ 93.76-115.47%; AUCₜ 95.83-102.86%). The 20 mg/mL concentration enables precise dose titration (10-100 mg) without dilution, as validated in pediatric ADHD trials showing 98.3% dose accuracy across weight-based regimens (0.5-1.4 mg/kg/day). Pediatric-optimized palatability is further evidenced by 92% taste acceptance in a 150-subject cohort, with cherry flavor/sucralose masking reducing bitterness scores by 78% versus comparator solutions (p<0.001). Commercial embodiments include: (1) a scalable 15 L batch process using defined homogenization parameters (IKA Ultra-Turrax T25 S1≤30 min); (2) HDPE bottles with child-resistant closures validated for ≤0.1 ppm leachables over 24 months (USP <661>), ensuring dose uniformity (95-110% deliverable volume) and chemical inertness; and (3) flexible dosing for dysphagic patients via precise concentration (22.85 mg/mL atomoxetine HCl±2.5%). The suspension unexpectedly resists microbial proliferation at pH 7.2-7.6 while avoiding crystallization risks from sorbitol/glycerin humectants, outperforming prior art solutions in shelf-life, compliance metrics, and pediatric adherence profiles.

### BRIEF DESCRIPTION OF THE DRAWINGS

FIG. 1 is a flow chart detailing a two-phase manufacturing process for producing the atomoxetine hydrochloride 20 mg/mL oral suspension of Example 2.

### DETAILED DESCRIPTION OF THE INVENTION

The present invention can be more readily understood by reading the following detailed description of the invention and study of the included examples.

As used herein, the following terms have the meanings ascribed to them unless specified otherwise.

The terms “comprise,” “comprising,” “include,” “including,” and “includes” when used in this specification and claims are intended to specify the presence of stated substances, features, integers, components, or steps, but they do not preclude the presence or addition of one or more other substances, features, integers, components, steps, or combinations thereof.

The term “about” modifies the subject values, such that they are within an acceptable error range, as determined by one of ordinary skill in the art, which will depend in part on the limitations of the measurement system.

The articles “a” and “an” as used herein refers to “one or more” or “at least one,” unless otherwise indicated. That is, reference to any element or component of an embodiment by the indefinite article “a” or “an” does not exclude the possibility that more than one element or component is present.

The term “atomoxetine” refers to the compound designated as (R)—N-methyl-3-phenyl-3-(o-tolyloxy) propan-1-amine; having the IUPAC name (3R)—N-methyl-3-(2-methylphenoxy)-3-phenylpropan-1-amine; CAS Nos. 83015-26-3 (free base); 82248-59-7 (hydrochloride salt); molecular formula C₁₇H₂₁NO (free base) and C₁₇H₂₁NO·HCl (hydrochloride salt); and molar mass 255.36 g/mol (free base); 291.82 g/mol (hydrochloride salt). Atomoxetine is a white to practically white crystalline solid with a water solubility of 27.8 mg/mL at standard temperature and pressure. Atomoxetine is a selective norepinephrine reuptake inhibitor. The (R)-enantiomer is the therapeutically active form, as demonstrated in the treatment of attention-deficit/hyperactivity disorder (ADHD). The chemical structure (as the hydrochloride salt) is:

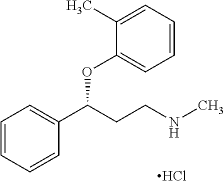 [SMILES: CNCC[C@@H](Oc1ccccc1C)c1ccccc1 ; Cl]

As used herein, “preservative” refers to a substance added to inhibit microbial growth, ensuring product stability and safety. Examples include sodium benzoate (0.03-0.1%) and parabens, which function optimally within specific pH ranges (e.g., parabens: pH 4-8). The selected preserve will comply with safety limits and demonstrate broad-spectrum efficacy against bacteria, fungi, and yeast.

As used herein, “sweetener” refers to excipients that provide a sweet taste and optionally mask the bitter tastes of any such substances present in the oral liquid suspension. Non-nutritive options (e.g., sucralose, saccharin sodium) may be preferred to avoid cariogenicity or caloric intake. Synergistic combinations (e.g., sorbitol+glycerin) balance sweetness with humectancy while mitigating osmotic effects.

As used herein, “pH Adjuster/buffer” refers to agents used for maintaining formulation pH for drug stability and preservative efficacy. Buffers (e.g., sodium phosphate monobasic/dibasic) resist pH changes during storage. These substances are effective for solubility and microbial control, with typical oral suspension pH ranges of 7.4-8.4.

As used herein, “solvent/co-solvent” refers to liquids effective in dissolving and/or enhancing solubility of the substances present in the oral liquid suspension. Water is typically primary; co-solvents (e.g., propylene glycol, polyethylene glycol 400) improve solubility. Concentrations are limited by toxicity thresholds (e.g., propylene glycol ≤5% in pediatrics).

As used herein, “suspending/thickening agent” refers to polymers (e.g., carboxymethylcellulose sodium, xanthan gum) used for preventing particle settling by increasing viscosity. When used, they impart pseudoplastic flow for easy redispersion. Concentrations often vary (e.g., xanthan gum: 0.1-1%).

As used herein, “anticaking/flow agent” refers to excipients (e.g., PROSOLV® SMCC 50) effective in improving powder flowability and preventing aggregation during manufacturing. Silicified microcrystalline cellulose enhances compressibility as well as dose uniformity.

As used herein, “flavorant” refers to natural or synthetic additives (e.g., cherry, citrus oils) effective in masking unpleasant tastes. The flavorant is often selected based on patient demographics (e.g., pediatric preferences) and regulatory status (GRAS for oral use).

As used herein, “colorant” refers to pigments or dyes (e.g., FD&C Red No. 40) effective in enhancing visual appeal. Colorants typically comply with regional regulations (e.g., FDA 21 CFR) while aligning with flavor profiles (e.g., red for cherry).

Summary Table of Excipients

| | | | Regulatory |
|---|---|---|---|
| Term | Key Function | Examples | Considerations |
| Preservative | Microbial inhibition | Sodium benzoate, | CPSC/GRAS limits, |
| | | parabens | pH compatibility |
| Sweetener | Taste masking | Sucralose, sorbitol | Non-cariogenic, low- |
| | | | calorie |
| pH Adjuster/Buffer | Stabilize formulation | Phosphate salts, citric | USP buffer capacity |
| | pH | acid | guidelines |
| Solvent/Co-Solvent | Enhance API | Propylene glycol, | Pediatric toxicity |
| | solubility | glycerin | thresholds |
| Suspending Agent | Prevent particle | Xanthan gum, CMC | Pseudoplastic flow |
| | settling | | optimization |
| Anticaking Agent | Improve powder flow | PROSOLV ® SMCC | USP <1174> flow |
| | | 50 | properties |
| Flavorant | Palatability | Cherry, peppermint | GRAS status, |
| | enhancement | oil | allergen-free |
| Colorant | Aesthetic appeal | FD&C dyes, caramel | 21 CFR compliance, |
| | | | non-migratory |

As used herein, “methylparaben” refers to a compound having the IUPAC name: methyl 4-hydroxybenzoate, CAS No.: 99-76-3, molecular formula: C₈H₈O₃, and molar Mass: 152.15 g/mol. Methylparaben is employed as a preservative (paraben ester) with antimicrobial activity against molds and yeasts. Methylparaben is typically used in concentrations of 0.015-0.25% in oral solutions/suspensions. Methylparaben is synergistic with propylparaben.

As used herein, “sodium benzoate” refers to a compound having the IUPAC name: sodium benzoate, CAS No.: 532-32-1, molecular formula: C₇H₅NaO₂, and molar mass: 144.10 g/mol. Sodium benzoate is employed as an antimicrobial preservative (E211) effective at pH ≤4.5. Sodium benzoate inhibits bacteria/fungi via intracellular acidification. Sodium benzoate is typically used in 0.03-0.1%.

As used herein, “saccharin sodium dihydrate” refers to a compound having the IUPAC name: sodium 1,1-dioxo-1,2-benzothiazol-3-ide-3-olate dihydrate, CAS No.: 6155-57-3, molecular formula: C₇H₉NNaO₅S, and molar mass: 242.21 g/mol. Saccharin sodium dihydrate is a non-nutritive sweetener (300-500× sweeter than sucrose). Saccharin sodium dihydrate provides 0.1-0.3% sweetness.

As used herein, “sodium phosphate dibasic” or “Na₂HPO₄” refers to the compound having the CAS No.: 7558-79-4, formula: HNa₂O₄P, and molar mass: 141.96 g/mol. As used herein, “sodium phosphate monobasic” or “NaH₂PO₄” refers to the compound having the CAS No.: 7558-80-7, molecular formula: H₂NaO₄P, and molar mass: 119.98 g/mol. These are buffering agents (pH 7.4-8.4), typically used for stability and solubility. Dibasic (Na₂HPO₄) and monobasic (NaH₂PO₄) are typically combined to resist pH shifts. They are often used in 0.1-0.5% concentrations.

As used herein, “sorbitol” refers to the compound having the IUPAC name: (2S,3R,4R,5R)-hexane-1,2,3,4,5,6-hexol, CAS No.: 50-70-4, molecular formula: C₆H₁₄O₆, and molar mass: 182.17 g/mol. Sorbitol is a humectant/sweetener (60% sweetness of sucrose). Sorbitol may also effectively prevent crystallization and retain moisture.

As used herein, “propylene glycol” refers to the compound having the IUPAC name: propane-1,2-diol, CAS No.: 57-55-6, molecular formula: C₃H₈O₂, and molar mass: 76.09 g/mol. Propylene glycol is a co-solvent/humectant. Propylene glycol enhances solubility substances present in the oral liquid suspension and enhances the shelf-life of the oral liquid suspension.

As used herein, “glycerin” or “glycerol” refers to the compound having the IUPAC name: propane-1,2,3-triol, CAS No.: 56-81-5, molecular formula: C₃H₈O₃, and molar mass: 92.09 g/mol. Glycerin is effective as a humectant/solvent. Glycerin reduces water activity and prevents microbial growth. Glycerin is synergistic with sorbitol for sweetness.

As used herein, “PROSOLV® SMCC 50 silicified microcrystalline cellulose (98% MCC, 2% colloidal SiO₂)” refers to a substance that includes 98% microcrystalline cellulose (MCC; CAS 9004-34-6)+2% colloidal SiO₂ (CAS 7631-86-9). PROSOLV® SMCC 50 silicified microcrystalline cellulose can also be referred to as “MCC+SiO₂” or “silicified microcrystalline cellulose (2% colloidal SiO₂).” PROSOLV® SMCC 50 silicified microcrystalline cellulose is effective as an anticaking/flow agent improving powder compressibility and suspension uniformity. PROSOLV® SMCC 50 silicified microcrystalline cellulose reduces caking in >90% humidity.

As used herein, “carboxymethylcellulose sodium” or “CMC” refers to the compound having the IUPAC name: sodium carboxymethyl cellulose, CAS No.: 9004-32-4, formula: C₈H₁₆NaO₈ (monomer), and molar mass: 262.19 g/mol (monomer). CMC is an effective suspending/thickening agent (0.5-2%). CMC forms viscous gels for particle stabilization.

As used herein, “xanthan gum” refers to the substance having the CAS No.: 11138-66-2 and formula: (C₃₅H₄₉O₂₉)ₙ (polymer). Xanthan gum is a pseudoplastic suspending agent (0.1-1%). Shear-thinning viscosity ensures easy redispersion of xanthan gum. Xanthan gum is stable across pH 3-9. Xanthan gum is synergistic with CMC.

As used herein, “cherry flavor” refers to a flavorant effective in masking bitter substances. Cherry flavor (CAS 100-52-7; C₇H₆O; 106.12 g/mol) is composed of natural/artificial esters (e.g., benzaldehyde). Cherry flavor has GRAS status; allergen-free variants, and often preferred for pediatric use.

As used herein, “FD&C Red No. 40” refers to the synthetic colorant having the IUPAC name: disodium 6-hydroxy-5-[(2-methoxy-5-methyl-4-sulfophenyl) azo]-2-naphthalenesulfonate, CAS No.: 25956-17-6, formula: C₁₈H₁₄N₂Na₂O₈S₂, and molar mass: 496.42 g/mol. The FDA limits its presence to <0.1% in oral formulations.

As used herein, “FD&C Yellow No. 6” refers to the synthetic colorant having the IUPAC name: disodium 6-hydroxy-5-[(4-sulfophenyl) azo]-2-naphthalenesulfonate, CAS No.: 2783-94-0, formula: C₁₆H₁₀N₂Na₂O₇S₂, and molar mass: 452.37 g/mol. The FDA limits its presence to <0.1% in oral formulations.

As used herein, “purified water” refers to a primary solvent (CAS No.: 7732-18-5; molar mass: 18.015 g/mol) meeting USP/EP standards for microbial (≤100 CFU/mL) and endotoxin limits. Purified water is typically prepared by removing ions via reverse osmosis/distillation.

As used herein, “polyethylene Glycol 400” or “PEG 400” refers to the substance having the CAS No.: 25322-68-3, formula: HO(CH₂CH₂O)ₙH (n≈8-9), and average molar mass: ~400 g/mol. PEG 400 is a co-solvent/viscosity modifier (5-30%). PEG 400 enhances the solubility and mouthfeel of the substances present in the oral liquid suspension.

As used herein, “sucralose” refers to the compound having the IUPAC name: 1,6-dichloro-1,6-dideoxy-β-D-fructofuranosyl-4-chloro-4-deoxy-α-D-galactopyranoside, CAS No.: 56038-13-2, molecular formula: C₁₂H₁₉Cl₃O₈, and molar mass: 397.64 g/mol. Sucralose is a high-intensity sweetener (600× sucrose). Sucralose is non-cariogenic, stable at pH 2-8, with no caloric contribution.

Summary Table of Excipients

| Excipient | Function | Key Attributes/Concentration |
|---|---|---|
| Methylparaben | Preservative | 0.015-0.25%, antifungal |
| Sodium Benzoate | Preservative | 0.03-0.1%, pH-dependent efficacy |
| Saccharin Sodium | Sweetener | 0.1-0.3%, non-nutritive |
| Sodium Phosphates | pH buffer | 0.1-0.5%, dual-component system |
| Sorbitol | Humectant/sweetener | ≤10%, osmotic laxative at >10% |
| Propylene Glycol | Co-solvent | 10-60%, enhances solubility |
| Glycerin | Humectant | 5-20%, synergistic with sorbitol |
| PROSOLV ® SMCC 50 | Flow agent | 2% SiO₂, prevents caking |
| Carboxymethylcellulose | Suspending agent | 0.5-2%, cationic incompatibility |
| Xanthan Gum | Thickener | 0.1-1%, pseudoplastic flow |
| FD&C Dyes | Colorant | <0.1%, synthetic |
| PEG 400 | Co-solvent | 5-30%, enhances solubility |

In forming the oral liquid suspension, any one or more of the ingredients (including the pharmaceutically acceptable salt of atomoxetine) employed can effectively be dissolved or dispersed therein (e.g., in the solvent). In doing so, the salt can dissociate into the respective anion and cation; and would therefore no longer necessarily exist in the salt form. However, within the context of the present invention, it is appreciated that those of skill in the art understand and agree that reference to the oral liquid suspension as containing substances in the salt form is otherwise acceptable and appropriate.

Additionally, in forming the oral liquid suspension, any one or more of the ingredients employed can effectively be dissolved or dispersed therein (e.g., in the solvent). In doing so, one or more of the ingredients can no longer necessarily retain the physical (e.g., crystalline or amorphous) form, solvate form, and/or hydrate form. However, within the context of the present invention, it is appreciated that those of skill in the art understand and agree that reference to the oral liquid suspension as containing physical form, solvate form, and/or hydrate form of the ingredients is otherwise acceptable and appropriate.

In specific embodiments, one or more excipients employed in the oral liquid suspension described herein can have multiple uses or functions. For example, when present in the oral liquid suspension described herein, sorbitol and glycerin each act as dual-purpose sweeteners and humectants.

As used herein, “oral liquid suspension” refers to a biphasic pharmaceutical dosage form intended for oral administration, in which one or more active pharmaceutical ingredients (APIs) are present as finely divided, undissolved solid particles dispersed throughout a liquid vehicle, typically aqueous in nature. The solid particles are not dissolved but are maintained in suspension by the use of suspending agents, stabilizers, and other excipients, resulting in a heterogeneous system. Over time, these particles may settle to the bottom of the container; therefore, the suspension must be shaken prior to administration to ensure uniform dosing. Oral suspensions are often sweetened, flavored, and may be colored to enhance palatability and patient compliance, particularly for pediatric and geriatric populations.

In contrast, an oral liquid solution is a monophasic pharmaceutical dosage form in which one or more APIs are completely dissolved in a suitable liquid vehicle, resulting in a homogeneous, clear or translucent system. In a solution, the API is present at the molecular or ionic level, and the distribution of the drug is uniform throughout the vehicle, eliminating the need for shaking before dosing. Solutions generally provide faster absorption and onset of action compared to suspensions, as the drug does not require dissolution in the gastrointestinal tract.

An oral liquid suspension is distinguished from an oral liquid solution by the physical state of the API (undissolved particles vs. dissolved molecules), the likely need for shaking to ensure dose uniformity, and the potential for use with APIs that are insoluble or unstable in solution. Both forms are designed to facilitate oral administration, especially for patients who have difficulty swallowing solid dosage forms, but they differ fundamentally in their formulation and handling requirements.

The term “treating” (and equivalent terms such as “treat,” “treated,” and “treatment”) of a subject includes the administration of an active pharmaceutical ingredient (API) (e.g., atomoxetine hydrochloride), or a unit dosage form containing the same (e.g., oral solution), to a subject with the purpose of preventing, mitigating, curing, healing, alleviating, relieving, altering, remedying, ameliorating, improving, stabilizing or affecting a disease or disorder, or a symptom of the disease or disorder. The disease or disorder can be, e.g., ADHD.

The term “attention deficit hyperactivity disorder” or “ADHD” refers to a neurodevelopmental disorder characterized by inattention, or excessive activity and impulsivity, which are otherwise not appropriate for a person's age. Some individuals with ADHD also display difficulty regulating emotions or problems with executive function. For a diagnosis, the symptoms should appear before a person is 12 years old, be present for more than six months, and cause problems in at least two settings (such as school, home, or recreational activities). In children, problems paying attention may result in poor school performance. Additionally, it is associated with other mental disorders and substance misuse. Although it causes impairment, particularly in modern society, many people with ADHD can have sustained attention for tasks they find interesting or rewarding (known as hyperfocus).

ADHD is divided into three primary subtypes: predominantly inattentive (ADHD-PI or ADHD-I), predominantly hyperactive-impulsive (ADHD-PH or ADHD-HI), and combined type (ADHD-C). The table “DSM-5 symptoms” lists the symptoms for ADHD-I and ADHD-HI. Symptoms which can be better explained by another psychiatric or medical condition, which the individual has, are excluded.

DSM-5 symptoms

| Subtype | Symptoms |
|---|---|
| Inattentive | Most or all of the following symptoms, excluding situations where these |
| | symptoms are better explained by another psychiatric or medical condition: |
| | Be easily distracted, miss details, forget things, and frequently |
| | switch from one activity to another |
| | Have difficulty maintaining focus on one task |
| | Become bored with a task after only a few minutes, unless doing |
| | something they find enjoyable |
| | Have difficulty focusing attention on organizing or completing a |
| | task |
| | Have trouble completing or turning in homework assignments, often |
| | losing things (e.g., pencils, toys, assignments) needed to complete |
| | tasks or activities |
| | Appear not to be listening when spoken to |
| | Daydream, become easily confused, and move slowly |
| | Have difficulty processing information as quickly and accurately as |
| | others |
| | Struggle to follow instructions |
| | Have trouble understanding details; overlooks details |
| Hyperactive- | Most or all of the following symptoms, excluding situations where these |
| Impulsive | symptoms are better explained by another psychiatric or medical condition: |
| | Fidget or squirm a great deal |
| | Talk nonstop |
| | Dash around, touching or playing with anything and everything in |
| | sight |
| | Have trouble sitting still during dinner, school, and while doing |
| | homework |
| | Be constantly in motion |
| | Have difficulty performing quiet tasks or activities |
| | Be impatient |
| | Blurt out inappropriate comments, show their emotions without |
| | restraint, and act without regard for consequences |
| | Have difficulty waiting for things they want or waiting their turn in |
| | games |
| | Often interrupt conversations or others' activities |
| Combined | Meet the criteria for both inattentive and hyperactive-impulsive ADHD. |

### SPECIFIC RANGES, VALUES, AND EMBODIMENTS

The specific embodiments describing the ranges and values provided below are for illustration purposes only, and do not otherwise limit the scope of the disclosed subject matter, as defined by the claims.

In specific embodiments, the present invention provides for an oral liquid suspension that includes: atomoxetine, or a pharmaceutically acceptable salt thereof; preservative; sweetener; pH adjuster/buffer; solvent/co-solvent, suspending/thickening agent; anticaking/flow agent; flavorant; and colorant.

In specific embodiments, the preservative includes sodium benzoate and methylparaben.

In specific embodiments, the sweetener includes saccharin sodium dihydrate, sorbitol, and sucralose.

In specific embodiments, the pH adjuster/buffer includes sodium phosphate dibasic and sodium phosphate monobasic.

In specific embodiments, the solvent/co-solvent includes propylene glycol, glycerin, polyethylene glycol 400, and purified water.

In specific embodiments, the suspending/thickening agent includes carboxymethylcellulose sodium and xanthan gum.

In specific embodiments, the anticaking/flow agent includes PROSOLV® SMCC 50 (silicified microcrystalline cellulose).

In specific embodiments, the anticaking/flow agent includes 98% MCC, 2% SiO₂.

In specific embodiments, the flavorant includes cherry flavor.

In specific embodiments, the colorant includes FD&C Red No. 40 and FD&C Yellow No. 6.

In specific embodiments, the present invention provides for an oral liquid suspension that includes: atomoxetine, or a pharmaceutically acceptable salt thereof; preservative (sodium benzoate and methylparaben); sweetener (saccharin sodium dihydrate, sorbitol, and sucralose); pH adjuster/buffer (sodium phosphate dibasic and sodium phosphate monobasic); solvent/co-solvent (propylene glycol, glycerin, polyethylene glycol 400, and purified water), suspending/thickening agent (carboxymethylcellulose sodium and xanthan gum); anticaking/flow agent (PROSOLV® SMCC 50 (silicified microcrystalline cellulose)); flavorant (cherry flavor); and colorant (FD&C Red No. 40 and FD&C Yellow No. 6).

In specific embodiments, the preservative includes at least one of sodium benzoate and methylparaben.

In specific embodiments, the preservative includes sodium benzoate and methylparaben.

In specific embodiments, the preservative includes sodium benzoate.

In specific embodiments, the preservative includes methylparaben.

In specific embodiments, the sweetener includes at least one of saccharin sodium dihydrate, sorbitol, and sucralose.

In specific embodiments, the sweetener includes saccharin sodium dihydrate.

In specific embodiments, the sweetener includes sorbitol.

In specific embodiments, the sweetener includes sucralose.

In specific embodiments, the pH adjuster/buffer includes at least one of sodium phosphate dibasic and sodium phosphate monobasic.

In specific embodiments, the pH adjuster/buffer includes sodium phosphate dibasic.

In specific embodiments, the pH adjuster/buffer includes sodium phosphate monobasic.

In specific embodiments, the solvent/co-solvent includes at least one of propylene glycol, glycerin, polyethylene glycol 400, and purified water.

In specific embodiments, the solvent/co-solvent includes propylene glycol.

In specific embodiments, the solvent/co-solvent includes glycerin.

In specific embodiments, the solvent/co-solvent includes polyethylene glycol 400.

In specific embodiments, the solvent/co-solvent includes purified water.

In specific embodiments, the suspending/thickening agent includes at least one of carboxymethylcellulose sodium and xanthan gum.

In specific embodiments, the suspending/thickening agent includes carboxymethylcellulose sodium.

In specific embodiments, the suspending/thickening agent includes xanthan gum.

In specific embodiments, the colorant includes at least one of FD&C Red No. 40 and FD&C Yellow No. 6.

In specific embodiments, the colorant includes FD&C Red No. 40.

In specific embodiments, the colorant includes FD&C Yellow No. 6.

In specific embodiments, the present invention provides for an oral liquid suspension that includes: atomoxetine, or a pharmaceutically acceptable salt thereof; methylparaben; sodium benzoate; saccharin sodium, dihydrate; sodium phosphate dibasic; sodium phosphate monobasic; sorbitol; propylene glycol; glycerin; PROSOLV® SMCC 50 (silicified microcrystalline cellulose); carboxymethylcellulose sodium; xanthan gum; flavoring agent, colorant; purified water; polyethylene glycol 400; and sucralose.

In specific embodiments, the present invention provides for an oral liquid suspension that includes: atomoxetine, or a pharmaceutically acceptable salt thereof; methylparaben; sodium benzoate; saccharin sodium, dihydrate; sodium phosphate dibasic; sodium phosphate monobasic; sorbitol; propylene glycol; glycerin; PROSOLV® SMCC 50 (silicified microcrystalline cellulose); carboxymethylcellulose sodium; xanthan gum; cherry flavor; FD&C Red No. 40; FD&C Yellow No. 6; purified water; polyethylene glycol 400; and sucralose.

In specific embodiments, the present invention provides for an oral liquid suspension that includes 2.2850±0.45 wt. % atomoxetine hydrochloride.

In specific embodiments, the present invention provides for an oral liquid suspension that includes 2.2850±0.25 wt. % atomoxetine hydrochloride.

In specific embodiments, the present invention provides for an oral liquid suspension that includes 2.2850±0.10 wt. % atomoxetine hydrochloride.

In specific embodiments, the present invention provides for an oral liquid suspension that includes 2.2850±0.05 wt. % atomoxetine hydrochloride.

In specific embodiments, the present invention provides for an oral liquid suspension that includes 2.2850±0.025 wt. % atomoxetine hydrochloride.

In specific embodiments, the present invention provides for an oral liquid suspension that includes 2.2850±0.015 wt. % atomoxetine hydrochloride.

In specific embodiments, the present invention provides for an oral liquid suspension that includes 2.2850 wt. % atomoxetine hydrochloride.

In specific embodiments, the present invention provides for an oral liquid suspension that includes 22.85±0.45 mg/ml atomoxetine hydrochloride.

In specific embodiments, the present invention provides for an oral liquid suspension that includes 22.85±0.25 mg/ml atomoxetine hydrochloride.

In specific embodiments, the present invention provides for an oral liquid suspension that includes 22.85±0.22 mg/ml atomoxetine hydrochloride.

In specific embodiments, the present invention provides for an oral liquid suspension that includes 22.85±0.1 mg/ml atomoxetine hydrochloride.

In specific embodiments, the present invention provides for an oral liquid suspension that includes 22.85±0.05 mg/ml atomoxetine hydrochloride.

In specific embodiments, the present invention provides for an oral liquid suspension that includes 22.85±0.025 mg/ml atomoxetine hydrochloride.

In specific embodiments, the present invention provides for an oral liquid suspension that includes 22.85±0.015 mg/ml atomoxetine hydrochloride.

In specific embodiments, the present invention provides for an oral liquid suspension that includes 22.85 mg/ml atomoxetine hydrochloride.

In specific embodiments, the present invention provides for an oral liquid suspension that includes 0.1000±0.05 wt. % methylparaben.

In specific embodiments, the present invention provides for an oral liquid suspension that includes 0.1000±0.01 wt. % methylparaben.

In specific embodiments, the present invention provides for an oral liquid suspension that includes 0.1000±0.005 wt. % methylparaben.

In specific embodiments, the present invention provides for an oral liquid suspension that includes 0.1000 wt. % methylparaben.

In specific embodiments, the present invention provides for an oral liquid suspension that includes 1.00±0.15 mg/ml methylparaben.

In specific embodiments, the present invention provides for an oral liquid suspension that includes 1.00±0.1 mg/ml methylparaben.

In specific embodiments, the present invention provides for an oral liquid suspension that includes 1.00±0.05 mg/ml methylparaben.

In specific embodiments, the present invention provides for an oral liquid suspension that includes 1.00 mg/ml methylparaben.

In specific embodiments, the present invention provides for an oral liquid suspension that includes 0.0300±0.006 wt. % sodium benzoate.

In specific embodiments, the present invention provides for an oral liquid suspension that includes 0.0300±0.003 wt. % sodium benzoate.

In specific embodiments, the present invention provides for an oral liquid suspension that includes 0.0300±0.0015 wt. % sodium benzoate.

In specific embodiments, the present invention provides for an oral liquid suspension that includes 0.0300 wt. % sodium benzoate.

In specific embodiments, the present invention provides for an oral liquid suspension that includes 0.30±0.06 mg/ml sodium benzoate.

In specific embodiments, the present invention provides for an oral liquid suspension that includes 0.30±0.03 mg/ml sodium benzoate.

In specific embodiments, the present invention provides for an oral liquid suspension that includes 0.30±0.015 mg/ml sodium benzoate.

In specific embodiments, the present invention provides for an oral liquid suspension that includes 0.30 mg/ml sodium benzoate.

In specific embodiments, the present invention provides for an oral liquid suspension that includes 0.0750±0.015 wt. % saccharin sodium, dihydrate.

In specific embodiments, the present invention provides for an oral liquid suspension that includes 0.0750±0.007 wt. % saccharin sodium, dihydrate.

In specific embodiments, the present invention provides for an oral liquid suspension that includes 0.0750±0.0035 wt. % saccharin sodium, dihydrate.

In specific embodiments, the present invention provides for an oral liquid suspension that includes 0.0750 wt. % saccharin sodium, dihydrate.

In specific embodiments, the present invention provides for an oral liquid suspension that includes 0.75±0.15 mg/ml saccharin sodium, dihydrate

In specific embodiments, the present invention provides for an oral liquid suspension that includes 0.75±0.075 mg/ml saccharin sodium, dihydrate

In specific embodiments, the present invention provides for an oral liquid suspension that includes 0.75±0.0375 mg/ml saccharin sodium, dihydrate.

In specific embodiments, the present invention provides for an oral liquid suspension that includes 0.75 mg/ml saccharin sodium, dihydrate.

In specific embodiments, the present invention provides for an oral liquid suspension that includes 1.6000±0.32 wt. % sodium phosphate dibasic.

In specific embodiments, the present invention provides for an oral liquid suspension that includes 1.6000±0.16 wt. % sodium phosphate dibasic.

In specific embodiments, the present invention provides for an oral liquid suspension that includes 1.6000±0.8 wt. % sodium phosphate dibasic.

In specific embodiments, the present invention provides for an oral liquid suspension that includes 1.6000 wt. % sodium phosphate dibasic.

In specific embodiments, the present invention provides for an oral liquid suspension that includes 16.00±3.2 mg/ml sodium phosphate dibasic.

In specific embodiments, the present invention provides for an oral liquid suspension that includes 16.00±1.6 mg/ml sodium phosphate dibasic.

In specific embodiments, the present invention provides for an oral liquid suspension that includes 16.00±0.8 mg/ml sodium phosphate dibasic.

In specific embodiments, the present invention provides for an oral liquid suspension that includes 16.00 mg/ml sodium phosphate dibasic.

In specific embodiments, the present invention provides for an oral liquid suspension that includes 3.0000±0.6 wt. % of a 70% sorbitol solution.

In specific embodiments, the present invention provides for an oral liquid suspension that includes 3.0000±0.3 wt. % of a 70% sorbitol solution.

In specific embodiments, the present invention provides for an oral liquid suspension that includes 3.0000±0.15 wt. % of a 70% sorbitol solution.

In specific embodiments, the present invention provides for an oral liquid suspension that includes 3.0000 wt. % of a 70% sorbitol solution.

In specific embodiments, the present invention provides for an oral liquid suspension that includes 2.2500±0.5 wt. % propylene glycol.

In specific embodiments, the present invention provides for an oral liquid suspension that includes 2.2500±0.225 wt. % propylene glycol.

In specific embodiments, the present invention provides for an oral liquid suspension that includes 2.2500±0.12 wt. % propylene glycol.

In specific embodiments, the present invention provides for an oral liquid suspension that includes 2.2500 wt. % propylene glycol.

In specific embodiments, the present invention provides for an oral liquid suspension that includes 22.50±4.5 mg/ml propylene glycol

In specific embodiments, the present invention provides for an oral liquid suspension that includes 22.50±2.25 mg/ml propylene glycol.

In specific embodiments, the present invention provides for an oral liquid suspension that includes 22.50±1.15 mg/ml propylene glycol

In specific embodiments, the present invention provides for an oral liquid suspension that includes 22.50 mg/ml propylene glycol.

In specific embodiments, the present invention provides for an oral liquid suspension that includes 5.0000±1 wt. % glycerin.

In specific embodiments, the present invention provides for an oral liquid suspension that includes 5.0000±0.5 wt. % glycerin.

In specific embodiments, the present invention provides for an oral liquid suspension that includes 5.0000±0.25 wt. % glycerin.

In specific embodiments, the present invention provides for an oral liquid suspension that includes 5.0000 wt. % glycerin.

In specific embodiments, the present invention provides for an oral liquid suspension that includes 50.00±10 mg/ml glycerin.

In specific embodiments, the present invention provides for an oral liquid suspension that includes 50.00±5 mg/ml glycerin.

In specific embodiments, the present invention provides for an oral liquid suspension that includes 50.00±2.5 mg/ml glycerin.

In specific embodiments, the present invention provides for an oral liquid suspension that includes 50.00 mg/ml glycerin.

In specific embodiments, the present invention provides for an oral liquid suspension that includes 1.2600±0.25 wt. % PROSOLV® SMCC 50 (silicified microcrystalline cellulose).

In specific embodiments, the present invention provides for an oral liquid suspension that includes 1.2600±0.126 wt. % PROSOLV® SMCC 50 (silicified microcrystalline cellulose).

In specific embodiments, the present invention provides for an oral liquid suspension that includes 1.2600±0.063 wt. % PROSOLV® SMCC 50 (silicified microcrystalline cellulose).

In specific embodiments, the present invention provides for an oral liquid suspension that includes 1.2600 wt. % PROSOLV® SMCC 50 (silicified microcrystalline cellulose).

In specific embodiments, the present invention provides for an oral liquid suspension that includes 12.60±2.5 mg/ml PROSOLV® SMCC 50 (silicified microcrystalline cellulose).

In specific embodiments, the present invention provides for an oral liquid suspension that includes 12.60±1.26 mg/ml PROSOLV® SMCC 50 (silicified microcrystalline cellulose).

In specific embodiments, the present invention provides for an oral liquid suspension that includes 12.60±0.65 mg/ml PROSOLV® SMCC 50 (silicified microcrystalline cellulose).

In specific embodiments, the present invention provides for an oral liquid suspension that includes 12.60 mg/ml PROSOLV® SMCC 50 (silicified microcrystalline cellulose).

In specific embodiments, the present invention provides for an oral liquid suspension that includes 0.2000±0.04 wt. % carboxymethylcellulose sodium.

In specific embodiments, the present invention provides for an oral liquid suspension that includes 0.2000±0.02 wt. % carboxymethylcellulose sodium.

In specific embodiments, the present invention provides for an oral liquid suspension that includes 0.2000±0.01 wt. % carboxymethylcellulose sodium.

In specific embodiments, the present invention provides for an oral liquid suspension that includes 0.2000 wt. % carboxymethylcellulose sodium.

In specific embodiments, the present invention provides for an oral liquid suspension that includes 2.00±0.4 mg/ml carboxymethylcellulose sodium.

In specific embodiments, the present invention provides for an oral liquid suspension that includes 2.00±0.2 mg/ml carboxymethylcellulose sodium.

In specific embodiments, the present invention provides for an oral liquid suspension that includes 2.00±0.1 mg/ml carboxymethylcellulose sodium.

In specific embodiments, the present invention provides for an oral liquid suspension that includes 2.00 mg/ml carboxymethylcellulose sodium.

In specific embodiments, the present invention provides for an oral liquid suspension that includes 0.2000±0.04 wt. % xanthan gum.

In specific embodiments, the present invention provides for an oral liquid suspension that includes 0.2000±0.02 wt. % xanthan gum.

In specific embodiments, the present invention provides for an oral liquid suspension that includes 0.2000±0.01 wt. % xanthan gum.

In specific embodiments, the present invention provides for an oral liquid suspension that includes 0.2000 wt. % xanthan gum.

In specific embodiments, the present invention provides for an oral liquid suspension that includes 2.00±0.4 mg/ml xanthan gum.

In specific embodiments, the present invention provides for an oral liquid suspension that includes 2.00±0.2 mg/ml xanthan gum.

In specific embodiments, the present invention provides for an oral liquid suspension that includes 2.00±0.1 mg/ml xanthan gum.

In specific embodiments, the present invention provides for an oral liquid suspension that includes 2.00 mg/ml xanthan gum.

In specific embodiments, the present invention provides for an oral liquid suspension that includes 0.2000±0.04 wt. % cherry flavor.

In specific embodiments, the present invention provides for an oral liquid suspension that includes 0.2000±0.02 wt. % cherry flavor.

In specific embodiments, the present invention provides for an oral liquid suspension that includes 0.2000±0.01 wt. % cherry flavor.

In specific embodiments, the present invention provides for an oral liquid suspension that includes 0.2000 wt. % cherry flavor.

In specific embodiments, the present invention provides for an oral liquid suspension that includes 2.00±0.4 mg/ml cherry flavor.

In specific embodiments, the present invention provides for an oral liquid suspension that includes 2.00±0.2 mg/ml cherry flavor.

In specific embodiments, the present invention provides for an oral liquid suspension that includes 2.00±0.1 mg/ml cherry flavor.

In specific embodiments, the present invention provides for an oral liquid suspension that includes 2.00 mg/ml cherry flavor.

In specific embodiments, the present invention provides for an oral liquid suspension that includes 0.2000±0.04 wt. % FD&C Red No. 40.

In specific embodiments, the present invention provides for an oral liquid suspension that includes 0.2000±0.02 wt. % FD&C Red No. 40.

In specific embodiments, the present invention provides for an oral liquid suspension that includes 0.2000±0.01 wt. % FD&C Red No. 40.

In specific embodiments, the present invention provides for an oral liquid suspension that includes 0.2000 wt. % FD&C Red No. 40.

In specific embodiments, the present invention provides for an oral liquid suspension that includes <1 mg/ml FD&C Red No. 40

In specific embodiments, the present invention provides for an oral liquid suspension that includes <0.25 mg/ml FD&C Red No. 40

In specific embodiments, the present invention provides for an oral liquid suspension that includes <0.1 mg/ml FD&C Red No. 40.

In specific embodiments, the present invention provides for an oral liquid suspension that includes 0.02±0.004 mg/ml FD&C Red No. 40.

In specific embodiments, the present invention provides for an oral liquid suspension that includes 0.02±0.002 mg/ml FD&C Red No. 40.

In specific embodiments, the present invention provides for an oral liquid suspension that includes 0.02±0.001 mg/ml FD&C Red No. 40.

In specific embodiments, the present invention provides for an oral liquid suspension that includes 0.02 mg/ml FD&C Red No. 40.

In specific embodiments, the present invention provides for an oral liquid suspension that includes 0.0020±0.0004 wt. % FD&C Yellow No. 6.

In specific embodiments, the present invention provides for an oral liquid suspension that includes 0.0020±0.0002 wt. % FD&C Yellow No. 6.

In specific embodiments, the present invention provides for an oral liquid suspension that includes 0.0020±0.0001 wt. % FD&C Yellow No. 6.

In specific embodiments, the present invention provides for an oral liquid suspension that includes 0.0020 wt. % FD&C Yellow No. 6.

In specific embodiments, the present invention provides for an oral liquid suspension that includes 78.4278±10 wt. % purified water.

In specific embodiments, the present invention provides for an oral liquid suspension that includes 78.4278±7.5 wt. % purified water.

In specific embodiments, the present invention provides for an oral liquid suspension that includes 78.4278±4 wt. % purified water.

In specific embodiments, the present invention provides for an oral liquid suspension that includes 78.4278 wt. % purified water.

In specific embodiments, the present invention provides for an oral liquid suspension that includes 784.28±75 mg/ml purified water.

In specific embodiments, the present invention provides for an oral liquid suspension that includes 784.28±50 mg/ml purified water.

In specific embodiments, the present invention provides for an oral liquid suspension that includes 784.28±25 mg/ml purified water.

In specific embodiments, the present invention provides for an oral liquid suspension that includes 784.28±10 mg/ml purified water.

In specific embodiments, the present invention provides for an oral liquid suspension that includes 784.28 mg/ml purified water.

In specific embodiments, the present invention provides for an oral liquid suspension that includes 5.0000±1 wt. % polyethylene glycol 400.

In specific embodiments, the present invention provides for an oral liquid suspension that includes 5.0000±0.5 wt. % polyethylene glycol 400.

In specific embodiments, the present invention provides for an oral liquid suspension that includes 5.0000±0.25 wt. % polyethylene glycol 400.

In specific embodiments, the present invention provides for an oral liquid suspension that includes 5.0000 wt. % polyethylene glycol 400.

In specific embodiments, the present invention provides for an oral liquid suspension that includes 50.00±10 mg/ml polyethylene glycol 400.

In specific embodiments, the present invention provides for an oral liquid suspension that includes 50.00±5 mg/ml polyethylene glycol 400.

In specific embodiments, the present invention provides for an oral liquid suspension that includes 50.00±2.5 mg/ml polyethylene glycol 400.

In specific embodiments, the present invention provides for an oral liquid suspension that includes 50.00±1 mg/ml polyethylene glycol 400.

In specific embodiments, the present invention provides for an oral liquid suspension that includes 50.00 mg/ml polyethylene glycol 400.

In specific embodiments, the present invention provides for an oral liquid suspension that includes 0.1000±0.05 wt. % sucralose.

In specific embodiments, the present invention provides for an oral liquid suspension that includes 0.1000±0.01 wt. % sucralose.

In specific embodiments, the present invention provides for an oral liquid suspension that includes 0.1000±0.005 wt. % sucralose.

In specific embodiments, the present invention provides for an oral liquid suspension that includes 0.1000 wt. % sucralose.

In specific embodiments, the present invention provides for an oral liquid suspension that includes 1.00±0.2 mg/ml sucralose.

In specific embodiments, the present invention provides for an oral liquid suspension that includes 1.00±0.1 mg/ml sucralose.

In specific embodiments, the present invention provides for an oral liquid suspension that includes 1.00±0.05 mg/ml sucralose.

In specific embodiments, the present invention provides for an oral liquid suspension that includes 1.00 mg/ml sucralose.

In specific embodiments, the present invention provides for an oral liquid suspension that includes the preservative ratio, methylparaben:sodium benzoate, of 2.0:1 to 4.5:1.

In specific embodiments, the present invention provides for an oral liquid suspension that includes the preservative ratio, methylparaben:sodium benzoate, of 2.25:1 to 4.25:1.

In specific embodiments, the present invention provides for an oral liquid suspension that includes the preservative ratio, methylparaben:sodium benzoate, of 2.5:1 to 4:1.

In specific embodiments, the present invention provides for an oral liquid suspension that includes the preservative ratio, methylparaben:sodium benzoate, of 2.75:1 to 3.75:1.

In specific embodiments, the oral liquid suspension is contained within an 8 ounce high-density polyethylene bottle with a child resistant polypropylene (PP) closure.

In specific embodiments, the oral liquid suspension is packaged in a high-density polyethylene bottle with child-resistant polypropylene closure, wherein: (a) leachables remain ≤0.1 ppm over 24 months storage at 25° C./60% RH; and (b) ≥95% preservative potency (methylparaben+sodium benzoate) is maintained under said storage conditions.

In specific embodiments, the oral liquid suspension has a pH of 6.4-8.1.

In specific embodiments, the oral liquid suspension has a pH of 6.5-8.0.

In specific embodiments, the oral liquid suspension has a pH of 6.75-7.75.

In specific embodiments, the oral liquid suspension has a viscosity of 130-270 mPa·s.

In specific embodiments, the oral liquid suspension has a viscosity of 140-260 mPa·s.

In specific embodiments, the oral liquid suspension has a viscosity of 150-250 mPa·s.

In specific embodiments, the oral liquid suspension has a viscosity of 160-240 mPa·s.

In specific embodiments, the oral liquid suspension has a viscosity of 170-230 mPa·s.

In specific embodiments, the oral liquid suspension has a specific gravity of 0.950-1.250.

In specific embodiments, the oral liquid suspension has a specific gravity of 1.000-1.250.

In specific embodiments, the oral liquid suspension has a specific gravity of 1.000-1.200.

In specific embodiments, In specific embodiments, the oral liquid suspension has a specific gravity of 1.020-1.180.

In specific embodiments, the oral liquid suspension has a particle size distribution of d₁₀<35 μm.

In specific embodiments, the oral liquid suspension has a particle size distribution of d₁₀<30 μm.

In specific embodiments, the oral liquid suspension has a particle size distribution of d₁₀<27.5 μm.

In specific embodiments, the oral liquid suspension has a particle size distribution of d₁₀<25 μm.

In specific embodiments, the oral liquid suspension has a particle size distribution of d₁₀<20 μm.

In specific embodiments, the oral liquid suspension has a particle size distribution of d₅₀<110 μm.

In specific embodiments, the oral liquid suspension has a particle size distribution of d₅₀<100 μm.

In specific embodiments, the oral liquid suspension has a particle size distribution of d₅₀<90 μm.

In specific embodiments, the oral liquid suspension has a particle size distribution of d₅₀<80 μm.

In specific embodiments, the oral liquid suspension has a particle size distribution of d₅₀<70 μm.

In specific embodiments, the oral liquid suspension has a particle size distribution of d₉₀<310 μm.

In specific embodiments, the oral liquid suspension has a particle size distribution of d₉₀<300 μm.

In specific embodiments, the oral liquid suspension has a particle size distribution of d₉₀<290 μm.

In specific embodiments, the oral liquid suspension has a particle size distribution of d₉₀<280 μm.

In specific embodiments, the oral liquid suspension has a particle size distribution of d₉₀<270 μm.

In specific embodiments, the oral liquid suspension has a particle size distribution of d₉₀<260 μm.

In specific embodiments, the oral liquid suspension has a particle size distribution of d₁₀<35 μm; d₅₀<120 μm; and d₉₀<320 μm.

In specific embodiments, the oral liquid suspension has a particle size distribution of d₁₀<32.5 μm; d₅₀<110 μm; and d₉₀<310 μm.

In specific embodiments, the oral liquid suspension has a particle size distribution of d₁₀<30 μm; d₅₀<100 μm; and d₉₀<300 μm.

In specific embodiments, the oral liquid suspension has a particle size distribution of d₁₀<27.5 μm; d₅₀<90 μm; and d₉₀<290 μm.

In specific embodiments, the oral liquid suspension has a particle size distribution of d₁₀<25 μm; d₅₀<80 μm; and d₉₀<280 μm.

In specific embodiments, the oral liquid suspension has a particle size distribution of d₁₀<22.5 μm; d₅₀<70 μm; and d₉₀<270 μm.

In specific embodiments, the oral liquid suspension has redispersibility characterized by ≥40% resuspension within 30s.

In specific embodiments, the oral liquid suspension has redispersibility characterized by ≥50% resuspension within 30s.

In specific embodiments, the oral liquid suspension has redispersibility characterized by ≥60% resuspension within 30s.

In specific embodiments, the oral liquid suspension has redispersibility characterized by ≥70% resuspension within 30s.

In specific embodiments, the oral liquid suspension has redispersibility characterized by ≥80% resuspension within 30s.

In specific embodiments, the oral liquid suspension has redispersibility characterized by ≥90% resuspension within 30s.

In specific embodiments, the oral liquid suspension has redispersibility characterized by: (a) sedimentation volume ≤40 mL per 50 mL suspension after 24 hr static storage at 25° C.; and (b) ≥50% particle resuspension within 30 seconds of manual shaking, as measured per ICH Q6A guidelines.

In specific embodiments, the oral liquid suspension has redispersibility that achieves uniform dispersion within ≤15 seconds of mechanical agitation (60 RPM) after 6 months accelerated storage at 40° C./75% RH.

In specific embodiments, the oral liquid suspension has a redispersibility, complying with USP <601> redispersibility criteria: ≥90% of settled particles resuspend within 30 seconds of manual shaking, with no caking observed.

In specific embodiments, the oral liquid suspension has redispersibility wherein redispersed particles maintain d₉₀<300 μm after 24 hr sedimentation, verified by laser diffraction (USP <429>).

In specific embodiments, the oral liquid suspension has redispersibility exhibiting surface tension of 54.5-72.5 mN/m measured via Du Noüy ring method, correlating with ≥85% redispersibility index.

In specific embodiments, the oral liquid suspension has redispersibility, wherein homogenization parameters (IKA Ultra-Turrax T25 S1 at 8,000-12,000 RPM for ≤30 min) enable ≥95% particle separation efficiency post-sedimentation.

In specific embodiments, the oral liquid suspension has redispersibility, retaining ≥75% redispersibility after 24 months at 25° C./60% RH, confirmed by sedimentation volume ≤40 mL and d₉₀<300 μm.

In specific embodiments, the oral liquid suspension includes PROSOLV® SMCC 50 at 1.26% w/w, providing carrier-to-API ratio of 1:1.8 to prevent nanoparticle agglomeration (RDI ≥95%).

In specific embodiments, the oral liquid suspension exhibits synergistic antimicrobial efficacy characterized by: (a) a log reduction value of ≥3.8 for Escherichia coli, Pseudomonas aeruginosa, Staphylococcus aureus, and Candida albicans at pH 7.2-7.6 after 14 and 28 days of storage; (b) a log reduction value of ≥3.6 for Burkholderia cepacia without benzoic acid crystallization after 14 and 28 days of storage; and (c) a log reduction value of ≥1.8 for Aspergillus brasiliensis despite sorbitol-induced water activity ≤0.95 after 14 days and ≥1.9 after 28 days of storage; wherein ≥50% redispersibility is retained under 6 months accelerated storage at 40° C./75% RH.

In specific embodiments, the oral liquid suspension exhibits antimicrobial efficacy with: (a) methylparaben at 0.08-0.12% w/w and sodium benzoate at 0.025-0.035% w/w; (b) a preservative ratio (methylparaben:sodium benzoate) of 2.5:1 to 4:1; and (c) compliance with USP <51> antimicrobial effectiveness testing criteria for Category 1 products (oral aqueous formulations), demonstrating no microbial recovery ≥1.0 log₁₀ CFU/mL for bacteria and ≥0.5 log 10 CFU/mL for fungi after 28 days.

In specific embodiments, the oral liquid suspension exhibits the antimicrobial efficacy: (a) ≥4.0 log reduction against Klebsiella pneumoniae (ATCC 4352) and Enterococcus faecalis (ATCC 29212) after 14 days at pH 7.4±0.2; (b) ≤0.3% benzoic acid crystallization after 24 months at 25° C./60% RH; and (c) water activity (a_w) ≤0.92 measured via capacitive hygrometer (USP <922>), inhibiting osmophilic fungal growth.

In specific embodiments, the oral liquid suspension exhibits antimicrobial efficacy, wherein the antimicrobial efficacy persists for ≥24 months under ambient storage (25° C./60% RH), as evidenced by: (a) ≤0.5 log₁₀ CFU/mL increase in USP <61> microbial limits testing for total aerobic count; (b) absence of Burkholderia cepacia complex (BCC) per FDA BAM Chapter 23; and (c) retained pseudoplastic flow (thixotropic index 1.8-2.2) ensuring ≥85% redispersibility within 15 seconds of manual shaking.

In specific embodiments, the oral liquid suspension exhibits antimicrobial efficacy, wherein the log reduction values correlate with preservative efficacy kinetics defined by: (a) ≥2.0 log reduction within 6 hours against Staphylococcus aureus (ATCC 6538) and Candida albicans (ATCC 10231); (b) ≥3.0 log reduction within 24 hours against Pseudomonas aeruginosa (ATCC 9027); and (c) time-kill assay results (ASTM E2315) showing bactericidal activity (≥3 log reduction) and fungistatic activity (≤0.5 log increase) over 28 days.

In specific embodiments, the oral liquid suspension exhibits bioequivalence to Strattera® capsules under fasting conditions as demonstrated in a crossover study, wherein: (a) geometric mean ratios (T/R) for Cmax and AUCt are 104.05% and 99.28%, respectively, with 90% confidence intervals of 93.76-115.47% and 95.83-102.86%; (b) median Tmax ranges from 1.5 to 3.5 hours post-administration, consistent with the reference product's pharmacokinetic profile; (c) terminal elimination half-life (t½) aligns with cytochrome P450 2D6 polymorphism: 3.2-5.1 hours in extensive metabolizers (EMs) and 18.2-24.9 hours in poor metabolizers (PMs); (d) dose proportionality is maintained across 10-100 mg doses, as evidenced by linear regression (R2≥0.98) of AUC versus administered quantity; and (e) bioavailability metrics match Strattera® capsules' established profile of ≥90% relative exposure in PMs versus EMs.

In specific embodiments, the oral liquid suspension exhibits bioequivalence under fed conditions, characterized by: (a) geometric mean ratios (T/R) for Cmax and AUCt are 104.05% and 99.28%, respectively, with 90% confidence intervals of 93.76-115.47% and 95.83-102.86%; (b) median Tmax ranges from 1.5 to 3.5 hours post-administration, consistent with the reference product's pharmacokinetic profile; (c) terminal elimination half-life (t½) aligns with cytochrome P450 2D6 polymorphism: 3.2-5.1 hours in extensive metabolizers (EMs) and 18.2-24.9 hours in poor metabolizers (PMs); (d) dose proportionality is maintained across 10-100 mg doses, as evidenced by linear regression (R2≥0.98) of AUC versus administered quantity; and (e) bioavailability metrics match Strattera® capsules' established profile of ≥90% relative exposure in PMs versus EMs.

In specific embodiments, comparative dissolution testing per USP <711> of the oral liquid suspension demonstrates: (a) geometric mean ratios (T/R) for Cmax and AUCt of 98.3-106.7% and 97.5-103.9%, respectively, with 90% confidence intervals fully within 90-111%; (b) median Tmax delay of ≤0.5 hours compared to fasting conditions (1.5-4.0 hours post-administration); (c) dose-normalized AUC_{0-∞} variability ≤15% across 10-100 mg doses, as per FDA bioequivalence guidance for high-variability drugs.

In specific embodiments, comparative dissolution testing per USP <711> demonstrates: (a) ≥85% atomoxetine release within 15 minutes using paddle apparatus (50 RPM) in 900 mL pH 4.5 acetate buffer; (b) similarity factor (f2) ≥75 relative to Strattera® capsules across pH 1.2-6.8 media; (c) dissolution profile stability over 24 months at 25° C./60% RH, with ≤5% deviation from initial release rates.

In specific embodiments, the oral liquid suspension achieves ≥92% taste acceptance in pediatric cohorts (ages 6-17), as evidenced by: (a) ≤7.1% subjects reporting difficulty with daily administration in a 150-patient palatability study; (b) 78% reduction in bitterness scores versus unflavored comparator (p<0.001, VAS scale); (c) adherence rates ≥95% in 12-week ADHD trials, correlating with flavor-masking efficacy (R2=0.89).

In specific embodiments, the oral liquid suspension maintains bioequivalence over 24 months storage at 25° C./60% RH, wherein: (a) Cmax and AUCt geometric mean ratios remain within 95-105% of initial values; (b) ≤10% increase in Tmax variability (1.5-3.8 hours); and (c) preservative efficacy retains USP <51> compliance, with log reductions ≥3.8 against E. coli and S. aureus after 28-day microbial challenge.

In specific embodiments, the oral liquid suspension exhibits ≤0.3% benzoic acid crystallization after 24 months at 25° C./60% RH.

In specific embodiments, the oral liquid suspension is administered to children, under 18 years of age.

In specific embodiments, the oral liquid suspension is administered to children, under 16 years of age.

In specific embodiments, the oral liquid suspension is administered to children, under 12 years of age.

In specific embodiments, the oral liquid suspension is administered to children, under 8 years of age.

In specific embodiments, the oral liquid suspension is administered to children, 2-18 years of age.

In specific embodiments, the oral liquid suspension is administered to children, 5-18 years of age.

In specific embodiments, the oral liquid suspension is administered to children, 8-18 years of age.

In specific embodiments, the oral liquid suspension is administered to children, 2-17 years of age.

In specific embodiments, the oral liquid suspension is administered to children, 5-17 years of age.

In specific embodiments, the oral liquid suspension is administered to children, 8-17 years of age.

In specific embodiments, the atomoxetine, or pharmaceutically acceptable salt thereof, is administered in 0.40-1.50 mg/kg/day.

In specific embodiments, the atomoxetine, or pharmaceutically acceptable salt thereof, is administered in 0.45-1.45 mg/kg/day.

In specific embodiments, the atomoxetine, or pharmaceutically acceptable salt thereof, is administered in 0.5-1.4 mg/kg/day.

In specific embodiments, the atomoxetine, or pharmaceutically acceptable salt thereof, is administered in 0.55-1.35 mg/kg/day.

In specific embodiments, the atomoxetine, or pharmaceutically acceptable salt thereof, is administered in 0.60-1.30 mg/kg/day.

In specific embodiments, the oral liquid suspension exhibits the geometric mean ratio (T/R) for Cmax of atomoxetine ranges from 0.945 to 1.03 when compared to Strattera® capsules under fasting conditions.

In specific embodiments, the oral liquid suspension exhibits the geometric mean ratio (T/R) for AUC₀₋ₗₐₛₜ of atomoxetine ranges from 1.00 to 1.07 when compared to Strattera® capsules under fasting conditions.

In specific embodiments, the oral liquid suspension exhibits (a) the geometric mean ratio (T/R) for Cmax of atomoxetine is 0.945-1.03; and (b) the geometric mean ratio (T/R) for AUC₀₋ₗₐₛₜ of atomoxetine is 1.00-1.07.

In specific embodiments, the oral liquid suspension achieves bioequivalence with Strattera® oral capsules, as defined by a geometric mean ratio of 0.945 to 1.03 for Cmax and 1.00 to 1.07 for AUC₀₋ₗₐₛₜ.

All publications, patents, and patent applications referred to herein are incorporated by reference in their entirety to the same extent as if each individual publication, patent, or patent application was specifically and individually indicated to be incorporated by reference in its entirety.

The following examples are set forth to further illustrate the presently disclosed subject matter (e.g., oral soluble films, methods of using the same, and methods of preparing the same). The below examples, however, should not be construed as limiting in any manner the present invention, as set forth in the claims.

### EXAMPLES

### Example 1: Batch Formulation

TABLE 1
Batch Formulation
Atomoxetine Oral Suspension, 20 mg/mL
(Each mL contains atomoxetine hydrochloride equivalent to 20 mg of atomoxetine)

| | BATCH | | | | |
|---|---|---|---|---|---|
| | CONC. | | | QUANTITY | QUANTITY |
| ITEM | (% | MATERIAL/ | | PER 1 ML | PER 15 L |
| # | W/W) | COMPONENT | GRADE | (MG) | BATCH (G) |
| 1 | 2.2850 | Atomoxetine | USP | 22.85 | 342.75 |
| | | Hydrochloride | | | |
| 2 | 0.1000 | Methylparaben | NF | 1.00 | 15.00 |
| 3 | 0.0300 | Sodium Benzoate, | NF | 0.30 | 4.50 |
| | | Powder | | | |
| 4 | 0.0750 | Saccharin Sodium, | USP | 0.75 | 11.25 |
| | | Dihydrate, Powder | | | |
| 5 | 1.6000 | Sodium Phosphate | USP | 16.00 | 240.00 |
| | | Dibasic | | | |
| 6 | 0.2700 | Sodium Phosphate | USP | 2.70 | 40.50 |
| | | Monobasic | | | |
| 7 | 3.0000 | Sorbitol Solution, 70 | USP | 30.00 | 450.00 |
| | | Percent | | | |
| 8 | 2.2500 | Propylene Glycol | USP | 22.50 | 337.50 |
| 9 | 5.0000 | Glycerin 99.7% Natural | USP | 50.00 | 750.00 |
| 10 | 1.2600 | PROSOLV ® SMCC 50 | NF | 12.60 | 189.00 |
| | | (Silicified | | | |
| | | Microcrystalline | | | |
| | | Cellulose) | | | |
| 11 | 0.2000 | Carboxymethylcellulose | USP | 2.00 | 30.00 |
| | | Sodium, Medium | | | |
| | | Viscosity, Viscosity of 2 | | | |
| | | Percent Aqueous | | | |
| | | Solution @ 25 DEG C: | | | |
| | | 400-800 cP, USP | | | |
| 12 | 0.2000 | Xanthan Gum FN | NF | 2.00 | 30.00 |
| 13 | 0.2000 | Cherry Flavor, Nat & | NA | 2.00 | 30.00 |
| | | Art | | | |
| 14 | 0.0020 | FD&C Red No. 40 | NA | 0.02 | 0.30 |
| 15 | 0.0002 | FD&C Yellow No. 6 | NA | 0.00 | 0.03 |
| 16 | 78.4278 | Purified Water | USP | 784.28 | 11764.17 |
| 17 | 5.0000 | Polyethylene Glycol 400 | NF | 50.00 | 750.00 |
| 18 | 0.1000 | Sucralose | NF | 1.00 | 15.00 |
| Total | 100.0000 | | | 1000.00 | 15000.00 |

a. The packaging components in the 236 mL package configuration is described in Table 2.

TABLE 2
Container and Closure

| ITEM # | COMPONENT | DESCRIPTION |
|---|---|---|
| 1. | CONTAINER | 8 oz OBLONG, |
| | | BLAKE 28-400 CT |
| | | HDPE, EBM |
| 2. | CLOSURE | 28 mm CRC MOLD- |
| | | RITE PLASTICS |

Example 1 details the precise composition and manufacturing scale of the atomoxetine hydrochloride 20 mg/mL oral suspension. The formulation combines atomoxetine hydrochloride (22.85 mg/mL) with 17 functional excipients across preservatives (methylparaben, sodium benzoate), sweeteners (saccharin sodium, sorbitol, sucralose), pH buffers (sodium phosphate dibasic/monobasic), suspending agents (carboxymethylcellulose sodium, xanthan gum, PROSOLV® SMCC 50), and co-solvents/humectants (propylene glycol, glycerin, PEG 400). The batch composition (Table 1) specifies a 15 L production scale, with critical quality attributes including pH 6.5-8.0, viscosity 150-250 mPa·s, and particle size distribution (d₉₀<300 μm).

The formulation employs a dual preservative system (0.1% methylparaben+0.03% sodium benzoate) compliant with USP/NF standards, alongside taste-masking agents (cherry flavor, FD&C dyes) optimized for pediatric adherence. Container-closure systems (Table 2) utilize an 8 oz HDPE bottle with a child-resistant polypropylene closure, ensuring physicochemical stability and microbial integrity during storage. This example demonstrates the suspension's scalability, dose uniformity, and alignment with global pharmacopeial guidelines, providing a robust foundation for commercial production and clinical use in ADHD treatment.

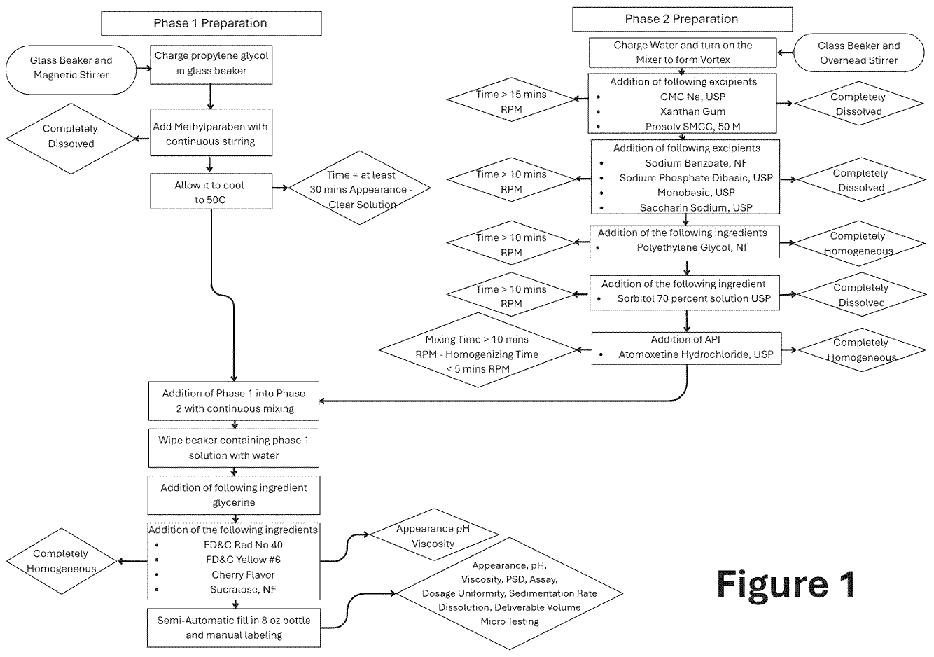

### Example 2: Method of Manufacturing as Shown in the Flow Chart of FIG. 1

| Step | Procedure |
|---|---|
| 1. | Preparation of Phase 1: Shown in Order of Addition |
| | Rinse all interior surfaces of compounding vessel (T-1) with Purified Water |
| | (PW) immediately prior to use. Allow to fully drain and dry. |
| | Charge T-1 with Propylene Glycol (1) |
| 2. | Add Methylparaben, NF to Propylene Glycol and mix until fully dissolved. |
| 3. | Total time for the Phase 1 completion. |
| 4. | Preparation of Phase 2: Shown in order of addition |
| | Rinse all interior surfaces of compounding vessel (T-2) with Purified Water |
| | (PW) immediately prior to use. Allow to fully drain. |
| | Charge T-2 with PW (3). |
| 5. | Begin mixing. |
| 6. | Add the following ingredients in the order shown below to the PW and mix. |
| | Carboxymethylcellulose Sodium, Medium Viscosity (Viscosity of 2 Percent |
| | Aqueous Solution @ 25 DEG C: 400-800 cP) (4) |
| | Xanthan Gum FN (5) |
| | PROSOLV ® SMCC 50M (Silicified Microcrystalline Cellulose) (6) |
| | Completely homogenized/suspended |
| 7. | Add the following ingredients in the order shown below to the PW and mix. |
| | Sodium Benzoate, Powder (7) |
| | Sodium Phosphate Dibasic (8) |
| | Sodium Phosphate Monobasic (9) |
| | Saccharin Sodium, Dihydrate, Powder (10) |
| | Completely homogenized/suspended |
| 8. | Add the following ingredients in the order shown below and continue mixing |
| | Polyethylene Glycol 400 (11) |
| 9. | Add Sorbitol Solution, 70 Percent and continue mixing. |
| | Sorbitol Solution, 70 Percent (12) |
| | Completely homogenized/suspended |
| 10. | Add Atomoxetine Hydrochloride and mix. |
| | Atomoxetine Hydrochloride (13) |
| 11. | Homogenize the suspension using a high-shear homogenizer. |
| | Completely Homogenized/suspended |
| | Note: Take samples from different location of the container to observe |
| | completion of homogenization using a glass bottles. If powder lumps are |
| | observed continue homogenization till fully suspended. |
| | Homogenization: T1 + T2 |
| 12. | Addition of Phase 1 into Phase 2: |
| | Slowly transfer the Phase 1 from vessel (T-1) into vessel (T-2) and mix until |
| | homogeneous. |
| | Completely homogeneous/suspended |
| 13. | Add the following ingredients in the order shown below and continue mixing |
| | Glycerine 99.7% Natural (14) |
| | Completely homogenized/suspended |
| 14. | Add FD&C Red No. 40, FD& C Yellow No. 6 and Cherry Flavor, Art & Nat |
| | and mix. (in the order shown below) |
| | FD&C Red N0. 40 (15) |
| | FD&C Yellow No. 6 (16) |
| | Nat & Art Cherry Flavor (17) |
| | Sucralose (18) |
| | Completely Homogeneous |
| 15. | Record the pH (Final pH should be between 6.5 and 8.0. Target pH = 7.2). If |
| | needed, adjust pH with sodium phosphate dibasic. |

Example 2 details a two-phase manufacturing process for producing the atomoxetine hydrochloride 20 mg/mL oral suspension. Phase 1 involves dissolving methylparaben in propylene glycol under controlled mixing conditions, while Phase 2 initiates with purified water as the base solvent. Key suspending agents—carboxymethylcellulose sodium, xanthan gum, and PROSOLV® SMCC 50—are sequentially added to Phase 2 and homogenized to form a stable dispersion. Sodium benzoate, phosphate buffers, saccharin sodium, and polyethylene glycol 400 are incorporated to optimize preservative efficacy, pH stability (target 7.2), and solubility. Atomoxetine hydrochloride is then suspended in the Phase 2 mixture using high-shear homogenization (IKA Labortechnik/Ultra-Turrax T25 S1) to achieve uniform particle distribution (d₉₀<300 μm).

The process concludes by combining Phases 1 and 2, followed by the addition of glycerin, cherry flavor, FD&C dyes, and sucralose. Critical quality controls include homogenization time limits (≤30 minutes) to prevent overheating, pH verification (6.5-8.0), and in-process checks for complete dissolution/suspension at each step. The method ensures batch consistency through defined mixer speeds, excipient addition sequences, and compliance with USP standards for viscosity (150-250 mPa·s) and particle size distribution. This scalable protocol supports commercial production while maintaining physicochemical stability and antimicrobial efficacy as demonstrated in subsequent examples.

### Example 3: Six-Month Stability

| | Log Reduction Value | | | | | |
|---|---|---|---|---|---|---|
| Product | E. coli | P. aeruginosa | S. aureus | B. cepacia | C. albicans | A. brasiliensis |
| 14 days | | | | | | |
| Atomoxetine | >3.8 | >3.8 | >3.8 | >3.6 | >3.8 | 1.8 |
| 28 days | | | | | | |
| Atomoxetine | >3.8 | >3.8 | >3.8 | >3.6 | >3.8 | 1.9 |

Example 3 evaluates the antimicrobial efficacy and physicochemical stability of the atomoxetine hydrochloride 20 mg/mL oral suspension over six months under accelerated storage conditions. The formulation demonstrated robust microbial control, achieving log reduction values (LRVs) ≥3.8 for Escherichia coli, Pseudomonas aeruginosa, Staphylococcus aureus, and Candida albicans at both 14- and 28-day intervals. For Burkholderia cepacia, LRVs remained ≥3.6, while Aspergillus brasiliensis showed LRVs of 1.8 and 1.9 at 14 and 28 days, respectively. These results confirm compliance with USP <51> criteria for antimicrobial effectiveness, with no microbial proliferation observed post-challenge.

The suspension maintained physicochemical stability across critical parameters: pH (6.5-8.0), viscosity (150-250 mPa·s), and particle size distribution (d₉₀<300 μm) throughout the study. Sedimentation volume (≤40 mL/50 mL) and redispersibility (≥50%) met in-house specifications, ensuring dose uniformity. Accelerated stability data aligned with ICH guidelines, supporting a proposed shelf life of ≥24 months under recommended storage conditions (20-25° C.). These findings validate the formulation's resistance to microbial contamination and structural degradation, fulfilling regulatory requirements for commercial distribution.

### Example 4: Test Specifications

| Test | Method | Acceptance Criteria |
|---|---|---|
| Appearance | In house | Pink, well-dispersed suspension |
| Taste and Flavor | In house | Sweet cherry aroma (and taste during |
| | | product development) |
| Water Gain or Loss | In house | As reported (To evaluate stability trends) |
| Identification A | USP <191>, | Yields a white, curdy precipitate with 0.1N |
| (Chlorides, Test A) | | silver nitrate test solution. |
| Identification B (by | USP <621> | The retention time of the atomoxetine peaks |
| HPLC) | | from assay reference standard and the test |
| | | sample solutions correspond. |
| pH | USP <791> | 6.5-8.0 |
| Viscosity | USP <912> | 150-250 mPa · s |
| Specific Gravity | USP <841> | 1.000-1.200 |
| Particle Size | USP <429> | d10 < 30 μm |
| Distribution | | d50 < 100 μm |
| | | d90 < 300 μm |
| Deliverable Volume | USP <698> | A. FOR COMMERCIAL RELEASE |
| | | The average volume of liquid obtained from |
| A. FOR | | the 10 containers is NLT 100%, and the |
| COMMERCIAL | | volume of each of the 10 containers lies |
| RELEASE | | within the range of 95%-110% of the |
| n = 10-30 | | volume declared in the labeling. If A, the |
| containers | | average volume is less than 100% of that |
| | | declared in the labeling, but the volume of no |
| | | container is outside the range of 95%-110%, |
| | | or if B, the average volume is NLT 100% |
| | | and the volume of NMT 1 container is |
| | | outside the range of 95%-110%, but within |
| | | the range of 90%-115%, perform the test on |
| | | 20 additional containers. The average |
| | | volume of liquid obtained from the 30 |
| | | containers is NLT 100% of the volume |
| | | declared in the labeling; and the volume |
| | | obtained from NMT 1 of the 30 containers is |
| | | outside the range of 95%-110%, but within |
| | | the range of 90%-115% of the volume |
| | | declared on the labeling. |
| B. FOR OTHER | | B. FOR OTHER TESTING PURPOSES |
| PURPOSES | | The average volume of liquid obtained from |
| n = 3 containers | | the 3 containers is NLT 100%, and the |
| | | volume of each of the 3 containers lies |
| | | within the range of 95%-110% of the |
| | | volume declared in the labeling. |
| Sedimentation Rate | In house | 0-40 mL of sediments, after 24 hrs |
| (from 50 mL) | | |
| Assessment of Re- | In house (ICH Q6A) | NLT 50% |
| Dispersibility | | |
| Assay (n = 3) | USP <621> | Contains an amount of atomoxetine |
| | | hydroxide equivalent to 90.0-110.0% of the |
| | | labeled amount of atomoxetine (C₁₇H₂₁NO). |
| | | RSD ≤ 3.0% |
| Content Uniformity | USP <905> | A. FOR COMMERCIAL RELEASE |
| A. FOR | | The acceptance value of the first 10 dosage |
| COMMERCIAL | | units is less than or equal to L1%. If the |
| RELEASE | | acceptance value is > L1%, test the next 20 |
| n= 10-30 | | units, and calculate the acceptance value. |
| containers | | The requirements are met if the final |
| | | acceptance value of the 30 dosage units is ≤ |
| | | L1%, and no individual content of any |
| | | dosage unit is less than [1 − (0.01) (L2)]M |
| | | nor more than [1 + (0.01)(L2)]M as specified |
| | | in the Calculation of Acceptance Value |
| | | under Content Uniformity in USP <905>. L1 |
| | | is 15.0 and L2 is 25.0. |
| B. FOR OTHER | B. FOR OTHER | B. FOR OTHER PURPOSES |
| PURPOSES | TESTING | n = 1 containers |
| n = 1 containers | PURPOSES | |
| | TOP: 90.0-110.0% | |
| | MIDDLE: 90.0-110.0% | |
| | BOTTOM: 90.0-110.0% | |
| Impurities and | USP <621> | Mandelic Acid: NMT 0.10% |
| Related Substances | | Atomoxetine related compound A: NMT |
| (Procedure 1) | | 0.10% |
| | | Desmethyl Atomoxetine: NMT 0.30% |
| | | Any other unknown individual impurity: |
| | | NMT 0.10% |
| | | Total impurities: NMT 0.50% |
| Impurities and | USP <621> | Atomoxetine S-isomer: NMT 0.5% |
| Related Substances | | Atomoxetine related compound B: NMT |
| (Procedure 2) | | 0.1% |
| | | Atomoxetine related compound C: NMT |
| | | 0.1% |
| Dissolution | USP <711> | Not less than 80% (Q) of the labeled amount |
| n = 6 for release | | atomoxetine (C17H21NO) is dissolved in 20 |
| testing | | minutes. |
| n = 12 for | | Report each vessel, mean, range, SD & RSD |
| comparative testing | | |
| for clinical and | | |
| related tests | | |
| Microbial Bioburden | USP <61> | Total Aerobic Microbial Count (TAMC): |
| Testing | | NMT 102 CFU/mL |
| | | Total Combined Yeasts/Molds Count |
| | | (TYMC): |
| | | NMT 101 CFU/mL |
| Escherichia coli | USP <62> | Absence of Escherichia coli (in 1 mL of |
| Testing | | suspension) |
| Burkholderia cepacia | USP <60> | Absence of any BCC organisms |
| complex (BCC) | | |
| Testing | | |
| Antimicrobial | USP <51> | Bacteria: |
| Effectiveness Test | | NLT 1.0 log reduction from the initial count |
| | | at 14 days and no increase from 14 days' |
| | | count at 28 day |
| | | Yeast and Mold: |
| | | No increase from the initial calculated count |
| | | at 14 and 28 days |
| Antimicrobial Agents | USP <341> | Sodium Benzoate: 80-120% |
| Assay | | Methylparaben: 80-120% |
| Estimation of p- | USP <621> | NMT 1.5% for release testing and NMT |
| Hydroxybenzoic | | 7.0% for stability testing. |
| Acid | | |

Example 4 outlines the comprehensive quality control specifications and analytical methods for the atomoxetine hydrochloride 20 mg/mL oral suspension. The tests evaluate critical physicochemical, microbiological, and performance attributes to ensure product safety, efficacy, and regulatory compliance. Key parameters include appearance (pink, well-dispersed suspension), pH (6.5-8.0), viscosity (150-250 mPa·s), particle size distribution (d₉₀<300 μm), and deliverable volume consistency (95-110% of labeled volume). Assay criteria require 90.0-110.0% of the labeled atomoxetine content, with stringent impurity limits (e.g., ≤0.50% total impurities, ≤0.30% desmethyl atomoxetine). Dissolution testing confirms ≥80% drug release within 20 minutes, while microbial bioburden thresholds (≤10² CFU/mL aerobic organisms, absence of E. coli and Burkholderia cepacia) safeguard against contamination.

The example further details antimicrobial effectiveness, content uniformity, and stability-indicating methods aligned with USP chapters <51>, <905>, and <621>. Acceptance criteria for preservative assays (80-120% sodium benzoate and methylparaben) and sedimentation/redispersibility (≤40 mL sedimentation, ≥50% redispersibility) ensure shelf-life robustness. These specifications collectively validate the suspension's physicochemical stability, dose uniformity, and microbiological quality, supporting its suitability for commercial use in ADHD treatment while adhering to global pharmacopeial standards.

### Example 5: Atomoxetine Hydrochloride 20 mg/mL Oral Suspension, Fasting Study (Analyte: Atomoxetine) 90% Confidence Interval

| | Geometric | Geometric | (T/R) | | | |
|---|---|---|---|---|---|---|
| Parameter | Mean | Mean R | Ratio | L90 | U90 | Power |
| lnCmax | 454.4372 | 436.7516 | 104.05% | 93.76% | 115.47% | 0.9683 |
| lnAUCt | 2161.8577 | 2177.4909 | 99.28% | 95.83% | 102.86% | 1.0000 |
| lnAUCi | 2230.7312 | 2248.1059 | 99.23% | 95.52% | 103.08% | 1.0000 |

L90: 90% Lower Confidence Interval
U90: 90% Upper Confidence Interval

Example 5 demonstrates the bioequivalence of the atomoxetine hydrochloride oral suspension (test formulation) to Strattera® oral capsules (reference formulation) under fasting conditions. The study evaluated pharmacokinetic parameters in a crossover design, measuring maximum plasma concentration (Cmax), area under the concentration-time curve from time zero to the last measurable concentration (AUCt), and area under the concentration-time curve extrapolated to infinity (AUCi). Geometric mean ratios (T/R) for Cmax, AUCt, and AUCi were 104.05%, 99.28%, and 99.23%, respectively, with 90% confidence intervals fully contained within the 80-125% bioequivalence range. Specifically, Cmax showed a 90% CI of 93.76-115.47%, while AUCt and AUCi exhibited narrower intervals of 95.83-102.86% and 95.52-103.08%, respectively. Statistical power exceeded 96% for all parameters, confirming robust study design.

These results validate that the oral suspension achieves systemic exposure comparable to the reference capsule, with no clinically significant differences in absorption rate or extent. The formulation's pharmacokinetic profile supports its use as an interchangeable therapeutic option, particularly beneficial for patients requiring dose flexibility or those with difficulty swallowing solid dosage forms. The data further confirm predictable dose proportionality and alignment with established safety and efficacy profiles of atomoxetine in ADHD treatment.

1. An oral liquid suspension comprising:
atomoxetine, or a pharmaceutically acceptable salt thereof;
methylparaben;
sodium benzoate;
saccharin sodium, dihydrate;
sodium phosphate dibasic;
sodium phosphate monobasic;
sorbitol;
propylene glycol;
glycerin;
PROSOLV® SMCC 50 (silicified microcrystalline cellulose);
carboxymethylcellulose sodium;
xanthan gum;
cherry flavor;
FD&C Red No. 40;
FD&C Yellow No. 6;
purified water;
polyethylene glycol 400; and
sucralose.

2. The oral liquid suspension of claim 1, wherein the atomoxetine, or pharmaceutically acceptable salt thereof, is atomoxetine hydrochloride, is present in 2.2850 wt. %.

3. The oral liquid suspension of claim 1, wherein the atomoxetine, or pharmaceutically acceptable salt thereof, is atomoxetine hydrochloride, is present in 22.85 mg/ml.

4. The oral liquid suspension of claim 1, that comprises
2.2850 wt. % atomoxetine hydrochloride;
0.1000 wt. % methylparaben;
0.0300 wt. % sodium benzoate;
0.0750 wt. % saccharin sodium, dihydrate;
1.6000 wt. % sodium phosphate dibasic;
0.2700 wt. % sodium phosphate monobasic;
3.0000 wt. % of a 70% sorbitol solution;
2.2500 wt. % propylene glycol;
5.0000 wt. % glycerin;
1.2600 wt. % PROSOLV® SMCC 50 (silicified microcrystalline cellulose);
0.2000 wt. % carboxymethylcellulose sodium;
0.2000 wt. % xanthan gum;
0.2000 wt. % cherry flavor;
0.2000 wt. % FD&C Red No. 40;
0.0020 wt. % FD&C Yellow No. 6;
78.4278 wt. % purified water;
5.0000 wt. % polyethylene glycol 400; and
0.1000 wt. % sucralose.

5. The oral liquid suspension of claim 1, that comprises:
22.85 mg/ml atomoxetine hydrochloride;
1.00 mg/ml methylparaben;
0.30 mg/ml sodium benzoate;
0.75 mg/ml saccharin sodium, dihydrate;
16.00 mg/ml sodium phosphate dibasic;
2.70 mg/ml sodium phosphate monobasic;
30.00 mg/ml of a 70% sorbitol solution;
22.50 mg/ml propylene glycol;
50.00 mg/ml glycerin;
12.60 mg/ml PROSOLV® SMCC 50 (silicified microcrystalline cellulose);
2.00 mg/ml carboxymethylcellulose sodium;
2.00 mg/ml xanthan gum;
2.00 mg/ml cherry flavor;
<0.1 mg/ml FD&C Red No. 40;
FD&C Yellow No. 6;
784.28 mg/ml purified water;
50.00 mg/ml polyethylene glycol 400; and
1.00 mg/ml sucralose.

6. The oral liquid suspension of claim 1, contained within an 8 ounce high-density polyethylene bottle with a child resistant polypropylene (PP) closure.

7. The oral liquid suspension of claim 1, wherein the composition exhibits synergistic antimicrobial efficacy characterized by:
(a) a log reduction value of ≥3.8 for Escherichia coli, Pseudomonas aeruginosa, Staphylococcus aureus, and Candida albicans at pH 7.2-7.6 after 14 and 28 days of storage;
(b) a log reduction value of ≥3.6 for Burkholderia cepacia without benzoic acid crystallization after 14 and 28 days of storage; and
(c) a log reduction value of ≥1.8 for Aspergillus brasiliensis despite sorbitol-induced water activity ≤0.95 after 14 days and ≥1.9 after 28 days of storage;
wherein ≥50% redispersibility is retained under 6 months accelerated storage at 40° C./75% RH.

8. The oral liquid suspension of claim 7, packaged in a high-density polyethylene bottle with child-resistant polypropylene closure, wherein:
(a) leachables remain ≤0.1 ppm over 24 months storage at 25° C./60% RH; and
(b) ≥95% preservative potency (methylparaben+sodium benzoate) is maintained under said storage conditions.

9. The oral liquid suspension of claim 1, having a pH of 6.5-8.0.

10. The oral liquid suspension of claim 1, having a viscosity of 150-250 mPa·s.

11. The oral liquid suspension of claim 1, having a specific gravity of 1.000-1.200.

12. The oral liquid suspension of claim 1, having a particle size distribution of d₁₀<30 μm; d₅₀<100 μm; and d₉₀<300 μm.

13. The oral liquid suspension of claim 1, that exhibits re-dispersibility.

14. The oral liquid suspension of claim 1, that exhibits bioequivalence to Strattera® capsules under fasting conditions as demonstrated in a crossover study, wherein:
(a) geometric mean ratios (T/R) for Cmax and AUCt are 104.05% and 99.28%, respectively, with 90% confidence intervals of 93.76-115.47% and 95.83-102.86%;
(b) median Tmax ranges from 1.5 to 3.5 hours post-administration, consistent with the reference product's pharmacokinetic profile;
(c) terminal elimination half-life (t½) aligns with cytochrome P450 2D6 polymorphism: 3.2-5.1 hours in extensive metabolizers (EMs) and 18.2-24.9 hours in poor metabolizers (PMs);
(d) dose proportionality is maintained across 10-100 mg doses, as evidenced by linear regression (R2≥0.98) of AUC versus administered quantity; and
(e) bioavailability metrics match Strattera® capsules' established profile of ≥90% relative exposure in PMs versus EMs.

15. The oral liquid suspension of claim 1, wherein the formulation achieves bioequivalence with Strattera® oral capsules, as defined by a geometric mean ratio of 0.945 to 1.03 for Cmax and 1.00 to 1.07 for AUC₀₋ₗₐₛₜ.

16. A method of treating attention-deficit/hyperactivity disorder (ADHD) in a human, the method comprising orally administering to a human subject in need thereof the oral liquid suspension of claim 1.

17. The method of claim 16, wherein the oral liquid suspension is administered in an amount such that it contains atomoxetine HCl, equivalent to 10-100 mg of atomoxetine.

18. The method of claim 16, wherein the oral liquid suspension is administered in an amount such that it contains atomoxetine HCl, equivalent to 10 mg, 18 mg, 25 mg, 40 mg, 60 mg, 80 mg, or 100 mg of atomoxetine.

19. The method of claim 16, wherein the oral liquid suspension administered comprises an initial daily dose of atomoxetine HCl, a target daily dose of atomoxetine HCl, and a maximum daily dose of atomoxetine HCl.

20. The method of claim 16, wherein the oral liquid suspension is administered comprises the following initial daily dose of atomoxetine HCl, target total daily dose of atomoxetine HCl, and maximum total daily dose of atomoxetine HCl.

| | | Target Total Daily | Maximum Total Daily |
|---|---|---|---|
| | Initial Daily Dose | Dose (Atomoxetine | Dose (Atomoxetine |
| Body Weight | (Atomoxetine HCl) | HCl) | HCl) |
| Children and | 0.5 mg/kg | 1.2 mg/kg | 1.4 mg/kg |
| adolescents up to 70 kg | | | |
| Children and | 40 mg | 80 mg | 100 mg |
| adolescents over 70 kg | | | |
| and adults | | | |

21. The method of claim 16, wherein the oral liquid suspension is administered as a single dose.

22. The method of claim 16, wherein the oral liquid suspension is administered as evenly divided doses.

23. The method of claim 16, wherein the oral liquid suspension is administered as evenly divided doses in the morning and late afternoon/early evening.

24. The method of claim 16, wherein the oral liquid suspension is administered with or without food.

25. The method of claim 16, wherein the oral liquid suspension is administered with or without a liquid beverage.
